(19) United States
(12) Patent Application Publication
(10) Pub. No.: US 2010/0010621 A1
(43) Pub. Date: Jan. 14, 2010

(54) STENT HAVING BIODEGRADABLE STENT STRUTS AND DRUG DEPOTS

(75) Inventor: Bjoern Klocke, Zurich (CH)

Correspondence Address: BARNES & THORNBURG LLP, Suite 1150, 3343 Peachtree Road, N.E., Atlanta, GA, 30326-1428, US

(73) Assignee: Biotronik VI Patent AG, Baar (CH)

(21) Appl. No.: 12/500,627

(22) Filed: Jul. 10, 2009

(30) Foreign Application Priority Data
Jul. 11, 2008 (DE) 10 2008 040 356.3

(51) Int. Cl. A61F 2/06

(52) U.S. Cl. 623/1.16; 623/1.42

(57) ABSTRACT

A stent comprising stent struts (1) of a biodegradable polymer material, drug depots (4) having at least one drug in the stent struts (1), at least one drug that treats the consequences of degradation of the stent struts (1) and sheathing of the drug depots (4) that varies over time, such that the drug delivery from the drug from the drug depots (4) is timed to coordinate with the mass degradation of the biodegradable polymer material of the stent struts (1).

### PRIORITY CLAIM

This patent application claims priority to German Patent Application No. 10 2008 040 356.3, filed Jul. 11, 2008, the disclosure of which is incorporated herein by reference in its entirety.

### FIELD

The present disclosure relates to a stent comprising stent struts made of a biodegradable polymer material and drug depots with at least one drug in the stent struts.

### BACKGROUND

According to the current state of the art with so-called polymer stents made of a biodegradable material, a single-phase stent basic body is provided, optionally having a thin layer releasing on its surface. This layer may contain an antiproliferative pharmaceutical ingredient, for example. It is also known that drug depots may be created within stent struts, e.g., from U.S. Patent Publication No. 2006/0224234. The stent disclosed therein should be explicitly free of polymer material, however.

U.S. Patent Publication No. 2004/0204750 discloses a drug-eluting stent for controlled drug delivery wherein the stent is manufactured on a polymer basis. These are not biodegradable stents. The drug depots that are provided in the stent struts are created in passages, for example, and their elution behavior can be defined by a continuous barrier over the stent struts. In this state of the art, there is no coordination with the degradation behavior of the stent struts.

Finally, U.S. Patent Publication No. 2004/0220660 discloses a bioabsorbable stent in i 5 which drug depots are again created in passages in the stent structure. These drug depots are closed with respect to the vascular lumen by a barrier layer while being open toward the vascular wall so that the drugs can escape from the drug depot. Here again, there is no correlation between the degradation behavior of the bioabsorbable stent and the drug delivery behavior from the depot.

Current research work in conjunction with bioabsorbable stents made of a biodegradable polymer material has shown that polymers degrade more slowly in vivo (and especially in elderly patients) than indicated by the prevailing research hypothesis in the past. Short-chain polymers, such as those obtained during the degradation process, remain in the tissue for a long period of time, even years, despite the loss of mechanical stability of the stent, and evidently become shortened only very slowly to monomers which can then be removed from the body by being transported through the surrounding body tissue. This massive formation of short-chain polymer regions in the tissue entails a substantial risk, so that at a certain point in time severe inflammation occurs, which is discussed in the literature under the catch phrase “tissue overload.” To some extent, there is also uncontrolled removal of polymer fragments by macrophages. Both effects can lead to undesirably high levels of inflammation which has a negative effect on the healing of tissue after stent implantation. In the worst case, polymer material may enter the bloodstream in comparatively large amounts and cause thromboses there.

In summary, with biodegradable polymer stents made of the usual materials, such as polyesters, which are degraded via the citrate cycle, there is thus the risk that despite continuous shortening of the polymer chain, the actual mass degradation takes place only with a so-called bulk release, i.e., a massive release of material in which the tolerance threshold of the surrounding tissue for the degradation products, in particular, for acids, may be exceeded. This may lead to an excessive inflammation reaction and possibly also to late thromboses.

### SUMMARY

The present disclosure describes several exemplary embodiments of the present invention.

One aspect of the present disclosure provides a stent, comprising a) a plurality of stent struts made of a biodegradable polymer material; and b) drug depots formed in the stent struts and having at least one drug in the stent struts, the drug depots further having a sheathing; and, (c) at least one drug that treats the consequences of degradation of the stent struts disposed in the drug depots, wherein the sheathing of the drug depots varies over time, such that the drug delivery from the drug depots is timed to coordinate with the mass degradation of the biodegradable polymer material of the stent struts.

The present disclosure provides a stent made of a biodegradable polymer material so that even late inflammation reactions in the tissue during the stent degradation process can still be controlled and there is no destruction of tissue with subsequent thromboses.

On the basis of this design of the sheathing of the drug depots, the drug to be eluted for the therapeutic purpose noted hereinabove then becomes free exactly when the degradation takes place and, in particular, when the problematical bulk release of the basic body of the stent takes place. It is thus possible to counteract the negative consequences of this event in a precisely targeted manner with the help of this drug.

### BRIEF DESCRIPTION OF THE DRAWINGS

Various aspects of the present disclosure are described hereinbelow with reference to the accompanying figures. Exemplary embodiments of the present disclosure are derived from the dependent claims. Additional features, details and advantages in this context are derived from the following description, which explains the exemplary embodiments on the basis of the accompanying diagrams in greater detail.

FIG. 1 shows a cross section view of a stent strut in a first exemplary embodiment;

FIG. 2 shows a cross section view of a stent strut in a second exemplary embodiment;

FIG. 3 shows a cross section view of a stent strut in a third exemplary embodiment;

FIG. 4 shows a perspective partial view of a stent strut in a fourth exemplary embodiment, and

FIG. 5 shows a longitudinal section view through the stent strut shown in FIG. 4.

### DETAILED DESCRIPTION

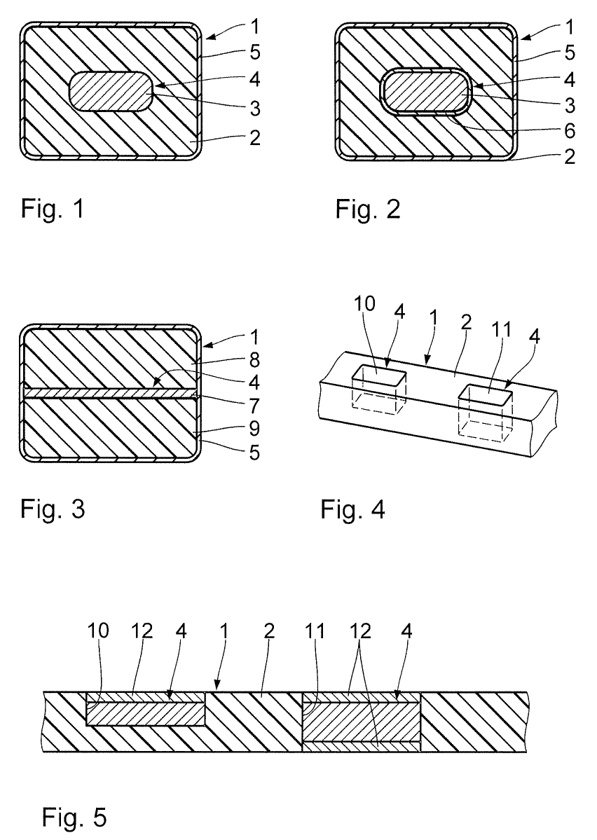

The stent strut 1 of a first exemplary embodiment shown in FIG. 1 consists of an elongated stent basic body 2 which has an approximately rectangular cross section which essentially determines the mechanical properties of the stent. The stent basic body consists of a biodegradable polymer material such as PLLA.

At its core, the stent basic body 2 is provided with a core 3 in the form of a continuous tube in the longitudinal direction containing a drug depot 4. The stent basic body 2 thus completely surrounds the drug depot 4. The latter comprises one or more drugs, such as anti-inflammatory drugs, e.g., paclitaxel or sirolimus, and their derivatives such as biolimus, everolimus, deforolimus, zotarolimus and others and/or a drug that promotes healing or anti-inflammatory and antithrombotic substances.

Antiproliferative, anti-inflammatory and/or antimycotic drugs may be selected from the following list, for example:
abciximab, acemetacin, acetylvismion B, aclarubicin, ademetionin, adriamycin, escin, afromoson, akagerine, aldesleukin, amidorone, aminoglutethemide, amsacrine, anakinra, anastrozole, anemonin, anopterine, antimycotics, antithrombotics, apocymarin, argatroban, aristolactam All, aristolochic acid, ascomycin, asparaginase, aspirin, atorvastatin, auranofin, azathioprin, azithromycin, baccatin, bafilomycin, basiliximab, bendamustine, benzocaine, berberine, betuline, betulinic acid, bilobol, bisparthenolidine, bleomycin, bombrestatin, boswellic acids and their derivatives, bruceanols A, B and C, bryophylline A, busulfan, antithrombin, bivalirudin, cadherine, camptothecin, capecitabine, o-carbamoylphenoxyacetic acid, carboplatin, carmustine, celecoxib, cepharanthine, cerivastatin, CETP inhibitors, chlorambucil, chloroquine phosphate, cictoxin, ciprofloxacin, cisplatine, cladribine, clarithromycin, colchicine, concanamycin, coumadin, C-type natriuretic peptide (CNP), cudraisoflavone A, curcumin, cyclophosphamide, cyclosporin A, cytarabine, dacarbazine, daclizumab, dactinomycin, dapsone, daunorubicin, diclofenac, 1,11-dimethoxycanthin-6-one, docetaxel, doxorubicin, dunaimycin, epirubicin, epothilones A and B, erythromycin, estramustine, etoposide, everolimus, filgrastim, fluroblastin, fluvastatin, fludarabine, fludarabine 5′-dihydrogen phosphate, fluorouracil, folimycin, fosfestrol, gemcitabine, ghalakinoside, ginkgol, ginkgolic acid, glycoside 1a, 4-hydroxyoxycyclophosphamide, idarubicin, ifosfamide, josamycin, lapachol, lomustine, lovastatin, melphalan, midecamycin, mitoxantrone, nimustine, pitavastatin, pravastatin, procarbazine, mitomycin, methotrexate, mercaptopurine, thioguanine, oxaliplatin, irinotecan, topotecan, hydroxycarbamide, miltefosine, pentostatin, pegasparase, exemestan, letrozole, formestan, SMC proliferation inhibitor 2w, mitoxanthrone, mycophenolate mofetil, c-myc-antisense, β-myc-antisense, β-lapachone, podophyllotoxin, podophyllic acid 2-ethyl hydrazide, molgramostim (rhuGM-CSF), peginterferon α-2b, lanograstim (r-HuG-CSF), macrogol, selectin (cytokine antagonist), cytokine inhibitors, COX-2 inhibitor, NF-kB, angiopeptin, monoclonal antibodies that inhibit muscle cell proliferation, bFGF antagonists, probucol, prostaglandins, 1-hydroxy-11-methoxycanthin-6-one, scopolectin, NO donors such as pentaerythrityl tetranitrate and syndnoeimines, S-nitroso derivatives, tamoxifen, staurosporin, β-estradiol, α-estradiol, estriol, estrone, ethynylestradiol, medroxyprogesterone, estradiol cypionate, estradiol benzoate, tranilast, kamebakaurin and other terpenoids which are used in oncotherapy, verapamil, tyrosine kinase inhibitors (tyrphostin), paclitaxel and its derivatives, such as 6-α-hydroxypaclitaxel, taxotere, carbon suboxide (MCS) and its macrocylcic oligomers, mofebutazone, lonazolac, lidocaine, ketoprofen, mefenamic acid, piroxicam, meloxicam, penicillamine, hydroxychloroquine, sodium aurothiomalate, oxaceprol, β-sitosterol, myrtecaine, polidocanol, nonivamide, levomenthol, ellipticine, D-24851 (Calbiochem), colcemid, cytochalasines A-E, indanocine, nocadazole, S 100 protein, bacitracin, vitronectin receptor antagonists, azelastine, guanidyl cyclase stimulator, tissue inhibitor of metal proteinase 1 and 2, free nucleic acids, nucleic acids incorporated into viral vectors, DNA and RNA fragments, plasminogen activator inhibitor 1, plasminogen activator inhibitor 2, antisense oligonucleotides, VEGF inhibitors, IGF-1, drugs from the antibiotic group, such as cefadroxil, cefazolin, cefaclor, cefotoxin, tobramycin, gentamycin, penicillins, such as dicloxacillin, oxacillin, sulfonamides, metronidazole, enoxoparin, desulfated and N-reacetylated heparin (commercially available as HEMOPARIN®), tissue plasminogen is activators, GpIIb/IIIa platelet membrane receptor, factor Xa inhibitor antibody, heparin, hirudine, r-hirudine, PPACK, protamine, prourokinase, streptokinase, warfarin, urokinase, vasodilators such as dipyramidol, trapidil, nitroprussides, PDGF antagonists such as triazolopyrimidine and seramine, ACE inhibitors such as captopril, cilazapril, lisinopril, enalapril, losartan, thioprotease inhibitors, prostacycline, vapiprost, interferons α, β and γ, histamine antagonists, serotonin blockers, apoptosis inhibitors, apoptosis regulators, e.g., p65, NF-kB or Bcl-xL antisense oligonucleotides, halofuginone, nifedipine, tocopherol tranilast, molsidomine, tea polyphenols, epicatechol gallate, epigallocatechol gallate, leflunomide, etanercept, sulfasalazine, etoposide, dicloxacillin, tetracycline, triamcinolone, mutamycin, procainimide, retinolic acid, quinidine, diisopyramide, flecainide, propafenone, sotolol, natural and synthetic steroids, such as inotodiol, maquiroside A, ghalakinoside, mansonin, strebloside, hydrocortisone, betamethasone, dexamethasone, non-steroidal anti-inflammatory drugs (NSAIDs), such as fenoprofen, ibuprofen, indomethacin, naproxen, phenylbutazone and other antiviral agents, such as acyclovir, ganciclovir and zidovudin, clotrimazole, flucytosine, griseofulvin, ketoconazole, miconazole, nystatin, terbinafin, antiprozoal agents such as chloroquine, mefloquine, quinine, as well as natural terpenoids such as hippocesculin, barringtogenol-C21-angelate, 14-dehydroagrostistachin, agroskerin, agrostistachin, 17-hydroxyagrostistachin, ovatodiolide, 4,7-oxycycloanisomelic acid, baccharinoids B1, B2, B3 and B7, tubeimoside, bruceantinoside C, yadanziosides N and P, isodeoxyelephantopine, tomenphantopines A and B, coronarin A, B, C and D, ursolic acid, hyptatic acid A, isoiridogermanal. maytenfoliol, effusantin A, excisanin A and B, longikaurin B, sculponeatin C, kamebaunin, leukamenin A and B, 13,18-dehydro-6-α-senecioyloxychaparrin, taxamairins A and B, regenilol, triptolide, and also cymarin, hydroxyanopterin, protoanemonin, cheliburin chloride, sinococulines A and B, dihydronitidine, nitidine chloride, 12β-hydroxypregnadien-3,20-dione, helenalin, indicine, indicine N-oxide, lasiocarpine, inotodiol, podophyllotoxin, justicidins A and B, larreatin, malloterin, mallotochromanol, isobutyrylmallotochromanol, maquiroside A, marchantin A, maytansin, lycoridicin, margetin, pancratistatin, liriodenin, bisparthenolidine, oxoushinsunin, periplocoside A, ursolic acid, deoxypsorospermin, psycorubin, ricin A, sanguinarin, manwuweic acid, methylsorbifoline, sphatheliachromene, stizophylline, mansonin, strebloside, dihydrousambarensin, hydroxyusambarin, strychnopentamine, strychnophylline, usambarin, usambarensin, liriodenin, oxoushinsunin, daphnoretin, lariciresinol, methoxylariciresinol, syringaresinol, sirolimus (rapamycin), somatostatin, tacrolimus, roxithromycin, troleandomycin, simvastatin, rosuvastatin, vinblastine, vincristine, vindesine, teniposide, vinorelbine, tropfosfamide, treosulfan, tremozolomide, thiotepa, tretinoin, spiramycin, umbelliferone, desacetylvismion A, vismion A and B, zeorin.

Preferred antiproliferative drugs include cytostatics, macrolide antibiotics, and/or statins. Suitable antiproliferative drugs that may also be mentioned include sirolimus (rapamycin), everolimus, pimecrolimus, somatostatin, tacrolimus, roxithromycin, dunaimycin, ascomycin, bafilomycin, erythromycin, midecamycin, josamycin, concanamycin, clarithromycin, troleandomycin, folimycin, cerivastatin, simvastatin, lovastatin, fluvastatin, rosuvastatin, atorvastatin, pravastatin, pitavastatin, vinblastine, vincristine, vindesine, vinorelbine, etoposide, teniposide, nimustine, carmustine, lomustine, cyclophosphamide, 4-hydroxyoxycyclophosphamide, estramustine, melphalan, betulinic acid, camptothecin, lapachol, β-lapachone, podophyllotoxin, betulin, tropfosfamide, podophyllic acid, 2-ethyl hydrazide, ifosfamide, chlorambucil, bendamustine, dacarbazine, busulfan, procarbazine, treosulfan, tremozolomide, thiotepa, daunorubicin, doxorubicin, aclarubicin, epirubicin, mitoxantrone, idarubicin, bleomycin, mitomycin, dactinomycin, methotrexate, fludarabine, fludarabine 5′-dihydrogen phosphate, mofebutazone, acemetacin, diclofenac, lonazolac, dapsone, o-carbamoylphenoxyacetic acid, lidocaine, ketoprofen, mefenamic acid, piroxicam, meloxicam, chloroquine phosphate, penicillamine, hydroxychloroquine, auranofin, sodium aurothiomalate, oxaceprol, celecoxib, β-sitosterol, ademetionin, myrtecaine, polidocanol, nonivamide, levomenthol, benzocaine, escin, cladribine, mercaptopurine, thioguanine, cytarabine, fluorouracil, gemcitabine, capecitabine, docetaxel, carboplatin, cisplatin, oxaliplatin, amsacrine, irinotecan, topotecan, hydroxycarbamide, miltefosine, pentostatin, aldesleukin, tretinoin, asparaginase, pegasparase, anastrozole, exemestan, letrozole, rormestan, aminoglutethemide, adriamycin, azithromycin, spiramycin, cepharanthine, SMC proliferation inhibitor 2w, epothilones A and B, mitoxanthrone, azathioprine, myco-phenolate mofetil, c-myc-antisense, b-myc-antisense selectin (cytokine antagonist) CETP inhibitor, cadherins, cytokine inhibitors, COX-2 inhibitor, NF-kB, angiopeptin, ciprofloxacin, camptothecin, fluroblastin, monoclonal antibodies that inhibit muscle cell proliferation, bFGF antagonists, probucol, prostaglandins, folic acid and derivatives, vitamins of the B series, vitamin D derivatives, such as calcipotriol and tacalcitol, thymosin-α-1, fumaric acid and its derivatives, such as dimethyl fumarate, IL-1β inhibitor, colchicine, NO donors such as pentaerthrityl tetranitrate and syndnoeimines, S-nitroso derivatives, tamoxifen, staurosporine, β-estradiol, α-estradiol, estrone, estriol, ethynylestradiol, fosfestrol, medroxyprogesterone, estradiol cypionates, estradiol benzoates, tranilast, kamebakaurin and other terpenoids that are used in oncotherapy, verapamil, tyrosine kinase inhibitors (tyrphostin), cyclosporin A, paclitaxel and its derivatives (6-α-hydroxypaclitaxel, baccatin, taxotere, etc.), synthetic as well as native macrocyclic oligomers of carbon suboxide (MCS) and its derivatives, molgramostim (rhuGM-CSF), peginterferon α-2b, lanograstim (r-HuG-CSF), filgrastim, macrogol, dacarbazine, basiliximab, daclizumab, ellipticine, D-24851 (Calbiochem), colcemide, cytochalasins A-E, indanocine, nocadazole, S 100 protein, PI-88, melanocyte stimulating hormone (α-MSH), bacitracin, vitronectin receptor antagonists, azelastine, guanidyl cyclase stimulator, tissue inhibitor of metal proteinase 1 and 2, free nucleic acids, nucleic acids incorporated into viral vectors, DNA and RNA fragments, plasminogen activator Inhibitor 1, plasminogen activator inhibitor 2, antisense oligonucleotide, VEGF inhibitors, IGF-1. In addition, the following substances from the group of antibiotics are also used: cefadroxil, cefazoline, cefaclor, cefotoxin, tobramycin, gentamycin. The following penicillins also have a positive influence on the postoperative phase: dicloxacillin, oxacillin, sulfonamides, metronidazole, antithrombotics, such as argatroban, aspirin, abciximab, synthetic antithrombin, bivalirudin, coumadin, enoxoparin, HEMOPARIN® (desulfated and N-reacetylated heparin), tissue plasminogen activator, GpIIb/IIIa platelet membrane receptor, factor Xa inhibitor, activated protein C, antibodies, heparin, hirudine, r-hirudine, PPACK, protamine, prourokinase, streptokinase, warfarin, urokinase, vasodilators such as dipyramidol, trapidil, nitroprusside, PDGF antagonists such as triazolopyrimidine and seramine, ACE inhibitors such as captopril, cilazapril, lisinopril, enalapril, losartan, thioprotease inhibitors, caspase inhibitors, apoptosis inhibitors, apoptosis regulators such as p65, NF-kB and Bcl-xL antisense oligonucleotides and prostacycline, vapiprost, α-, β- and γ-interferon, histamine antagonists, serotonin blocker, halofuginone, nifedipine, tocopherol, tranirast, molsidomine, tea polyphenols, epicatechol gallate, epigallocatechol gallate, boswellic acids and their derivatives, leflunomide, anakinra, etanercept, sulfasalazine, etoposide, dicloxacillin, tetracycline, triamcinolone, mutamycin, procainimide, retinolic acid, quinidine, disopyrimide, flecainide, propafenone, sotolol, amidorone. Other active ingredients include steroids (hydrocortisone, betamethasone, dexamethasone), non-steroidal anti-inflammatory drugs (NSAIDs) such as fenoprofen, ibuprofen, indomethacin, naproxen, phenylbutazone and others. Antiviral agents such as acyclovir, ganciclovir and zidovudin can also be used. Various antimycotics are used in this area. Examples include clotrimazole, flucytosine, griseofulvin, ketoconazole, miconazole, nystatin, terbinafin. Antiprozoal agents such as chloroquine, mefloquine, quinine are likewise effective agents, as are the natural terpenoids, such as hippocaesculin, baiTingtogenol-C21-angelate, 14-dehydro-agrostistachin, agroskerin, agrostistachin, 17-hydroxyagrostistachin, ovatodiolides, 4,7-oxycycloanisomelic acid, baccharinoids B1, B2, B3, tubeimoside, bruceanols A, B, C, bruceantinoside C, yadanziosides N and P, isodeoxyelephantopin, tomenphantopins A and B, coronarin A, B, C and D, ursolic acid, hyptatic acid A, zeorin, isoiridogermanal, maytenfoliol, effusantin A, excisanins A and B, longikaurin B, sculponeatin C, kamebaunin, leukamenin A and B, 13,18-dehydro-6-α-senecioyloxychaparrin, 1,11-dimethoxycanthin-6-one, 1-hydroxy-11-methoxycanthin-6-one, scopolectin, taxamairins A and B, regenilol, triptolide, also cymarin, apocymarin, aristolochic acid, anopterin, hydroxyanopterin, anemonin, protoanemonin, berberin, cheliburin chloride, cictoxin, sinococulin, bombrestatins A and B, cudraisoflavone A, curcumin, dihydronitidine, nitidine chloride, 12-B-hydroxypregnadien-4,16-dien-3,20-dione, bilobol, ginkgol, ginkgolic acid, helenalin, indicine, indicine N-oxide, lasiocarpin, inotodiol, glycoside la, podophyllotoxin, justicidins A and B, larreatin, malloterin, mallotochromanol, isobutyrylmallotochromanol, maquiroside A, marchantin A, maytansin, lycoridicin, margetin, pancratistatin, liriodenin, oxoushinsunin, aristolactam All, bisparthenolidine, periplocoside A, ghalakinoside, ursolic acid, deoxypsorospermin, psycorubin, ricin A, sanguinarin, manwuweizic acid, methylsorbifolin, sphatheliachromene, stizophylline, mansonin, strebloside, akagerin, dihydrousambaraensin, hydroxyusambarin, strychnopentamine, strychnophylline, usambarin, usambarensin, berberin, liriodenin, oxoushinsunin, daphnoretin, lariciresinol, methoxylariciresinol, syringaresinol, umbelliferone, afromoson, acetylvismion B, desacetylvismion A, vismions A and B, other natural terpenoids, such as hippocaesculin, 14-dehydroagrostistachin, C-type natriuretic peptide (CNP), agroskerin, agrostistachin, 17-hydroxyagrostistachin, ovatodiolide, 4,7-oxycycloanisomelic acid, yadanziosides N and P, isodeoxyelephantopin, tomenphantopins A and B, coronarin A, B, C and D, ursolic acid, hyptatic acid A, zeorin, isoiridogermanal, maytenfoliol, effusantin A, excisanins A and B, longikaurin B, sculponeatin.

The drug(s) may be used alone as such or embedded in a bioabsorbable vehicle substance such as the polymer material of the stent basic body 2. Microencapsulation of the drug is also possible.

On the outside, the stent basic body 2 is coated with a drug delivery layer, namely a so-called drug-eluting layer 5, which may be formed from an antiproliferative substance that has an antiproliferative effect and is itself embedded in a vehicle material.

Because of the arrangement of the drug depot 4 as a core 3 in the stent basic body 2, the drugs in the drug depot 4 are released precisely when the stent basic body 2 has undergone degradation of its polymer chains to the extent that there is a reduction in the mass of polymer material. To this extent the stent basic body forming the sheathing of the drug depot 4 is a sheathing that is variable over time and is thus designed so that the drug is released from the drug depot 4 in a manner that is timed to coordinate with the bulk release of the biodegradable polymer material of the stent struts 1.

The second exemplary embodiment illustrated in FIG. 2 differs from that the embodiment shown in FIG. 1 only in that the core 3 is separated from the stent basic body 2 surrounding the drug depot 4 by a separating layer 6. This separating layer 6 thus serves as a macroscopic encapsulation of the drug depot 4 and serves as a diffusion brake for the drug. Therefore, the start of the drug delivery, which is delayed in time, can be controlled inasmuch as this is necessary for the drug delivery which is coordinated with the bulk release of stent strut material. The separating layer typically consists of a degradable polymer, e.g., polydioxanone, polyglycolide, polycaprolactone, polylactides (poly-L-lactide, poly-D,L-lactide and copolymers and blends such as poly(L-lactide-co-glycolide), poly(D,L-lactide-co-glycolide), poly(L-lactide-co-D,L-lactide), poly(L-lactide-co-trimethylene carbonate, triblock copolymers), polysaccharides (chitosan, levan, hyaluronic acid, heparin, dextran, cellulose, etc.), polyhydroxyvalerate, ethyl vinyl acetate, polyethylene oxide, poly-phosphorylcholine, fibrin or albumin.

It is also possible for the separating layer to be permeable and to consist of a permanent polymer, e.g., parylene, polypropylene, polyethylene, polyvinyl chloride, polymethyl methacrylate, polymethyl methacrylate, polytetrafluorethylene, polyvinyl alcohol, polyurethane, polybutylene terephthalate, silicone, polyphosphazene as well as their copolymers and blends or inorganic layers. In the case of a nondegradable separating layer, the layer thickness is to be selected to be preferably between approximately 0.2 μm and approximately 5 μm such that the encapsulated drug is still delivered with the correct kinetics. With regard to the remaining structure of the stent strut 1 according to FIG. 2, reference can be made to the statements made in conjunction with FIG. 1. Corresponding elements are labeled with the same reference numerals.

In the third exemplary embodiment according to FIG. 3, the drug depot 4 is designed as an intermediate layer 7 which is embedded between two layers 8, 9 of the stent basic body 2. In this design of the drug depot 4, it is advantageous if the drug is embedded in the intermediate layer 7 in a backing with slow diffusion.

The stent basic body 2 illustrated in FIG. 3 has an outer layer 5 as the drug-eluting layer.

Finally, FIGS. 4 and 5 illustrate a fourth exemplary embodiment of a stent basic body 2 in which the drug depots 4 are created in recesses 10 (see left part of FIG. 5) and/or passages 11 (see right part of FIG. 5). The open sides of these recesses 10 and/or passages 11 with the exposed surfaces of the drug depots 4 are sealed by a barrier layer 12, which has a high barrier effect and degrades only very slowly or not at all (e.g., parylene, BUMA, PLLA). Thus again, the stent basic body 2 itself forms the sheathing on the drug depot 4, which changes over time and can be opened with the bulk release of the stent polymer material and thus the drug can manifest its therapeutic effect. The drug in these drug depots 4 in the recesses 10 and/or passages 11 is in turn embedded in a vehicle.

Production of the stent struts 1 illustrated in FIGS. 1-5 with drug depots 4 can take place by conventional manufacturing methods such as a combination of machining, drilling and laser cutting for the removal of material to form the stent basic body 2, immersion and spraying for the application of layers and casting in a mold cavity to create large bodies, such as cylinders.

In FIG. 1, the stent basic body 2 can be extruded traditionally, e.g., for the case of a braided stent (so-called wall stent design). In the second step, the drug depot 4 is filled with a drug-solvent mixture (optionally with an additional vehicle substance) and the solvent is expelled by increasing the temperature, if necessary. This operation can be repeated to fill up the resulting cavities. As an alternative to step 2, filling the drug depot 4 with a powdered drug is also possible. The drug-eluting layer 5 may be applied, e.g., by a traditional spray method with solvent as the last step.

Alternatively, a core 3 may also be cast in liquid form in a mold cavity which can be opened. After cooling, the core is introduced into a larger cavity and the stent base body 2 is introduced, optionally by pressing.

The separating layer 6 in FIG. 2 can be inserted as an intermediate layer by coating the core 3 by a spray method before sheathing with the stent basic body 2.

A stent according to FIG. 3 can be assembled from laser-cut layers or may be prepared out of a sandwich tube by laser cutting.

The passages 11 and/or recesses 10 according to FIG. 4 can be created in the solid material by laser ablation. The barrier layers 12 on these recesses 10 or passages 11 can be applied step by step by a pipetting technique, for example.

All patents, patent applications and publications referred to herein are incorporated by reference in their entirety.

What is claimed is:

1. A stent, comprising:
a) a plurality of stent struts made of a biodegradable polymer material; and
b) drug depots formed in the stent struts and having at least one drug in the stent struts, the drug depots further having a sheathing; and,
(c) at least one drug that treats the consequences of degradation of the stent struts disposed in the drug depots,
wherein the sheathing of the drug depots varies over time, such that the drug delivery from the drug depots is timed to coordinate with the mass degradation of the biodegradable polymer material of the stent struts.

2. The stent of claim 1, wherein the drug is either an anti-inflammatory or a healing-promoting drug.

3. The stent of claim 1, wherein, to form the sheathing, the drug depots are created as a core in the stent struts.

4. The stent of claim 3, wherein the core is separated from the stent struts surrounding the core by either a degradable or a permeable separating layer.

5. The stent of claim 1, wherein the drug depots are created as an intermediate layer in the stent struts to form the sheathing.

6. The stent of claim 1, wherein, to form the sheathing, the drug depots are created either in recesses or in passages in the stent struts such that the open sides of the drug depots are sealed by a barrier layer that is either slowly degradable or not degradable at all.

7. The stent of claim 1, wherein each drug is either present alone, incorporated into a polymer vehicle material or microencapsulated.

8. The stent of claim 1, wherein the stent struts are provided with a drug-releasing outer layer on the outside of the stent struts.

9. The stent of claim 8, wherein the outer layer contains an antiproliferative substance embedded in a vehicle.
